(19) United States
(12) Patent Application Publication
(10) Pub. No.: US 2020/0376335 A1
(43) Pub. Date: Dec. 3, 2020

(54) WIRELESS COMMUNICATION OF SWIMMING METRICS

(71) Applicant: Splash Metrix, Inc., La Jolla, CA (US)

(72) Inventors: Eric A. WAN, La Jolla, CA (US); Jonathan SCHEFF, La Jolla, CA (US); Ian Muir MCNALLY, La Jolla, CA (US)

(21) Appl. No.: 16/888,406

(22) Filed: May 29, 2020

Related U.S. Application Data
(60) Provisional application No. 62/855,251, filed on May 31, 2019.

(51) Int. Cl. A63B 24/00; A63B 33/00; A63B 71/06; H04W 4/80; G06F 1/16

(52) U.S. Cl. CPC A63B 24/0006; A63B 24/0062; A63B 33/002; A63B 71/0622; H04W 4/80; A63B 2208/03; A63B 2024/0009; A63B 2071/0625; A63B 2225/50; A61B 2503/10; G06F 1/163

(57) ABSTRACT

A wireless communications system for determining and conveying swimming metrics to swimmers and triathletes during swimming activity. The swimming metric communication system comprises a smartwatch for determining swim metrics and a head-worn device with an integrated headphone, headphone jack, or wireless headphone transmitter. The smartwatch and head-worn device utilize a Bluetooth Low Energy (BLE) connection to communicate voiced swim metrics to the user. For example, various voiced data segments are preloaded on the head-worn device and associated with unique low-bit codes, respectively. The BLE connection is used to transmit certain low-bit codes to trigger respective voiced data segments. A transmission connection between the smartwatch and headphone, and vice-versa, is established when the user's hand is out of the water (recovery) and/or when the hand passes near the face underwater (pull). Once a connection is established and codes are received, the headphone decodes the sequence of codes and plays a respective voiced swimming metric without interruption to swimming activity. The head-worn device may include sensors for improving the accuracy of the swim metrics or for determining additional swim metrics, which are communicated to the smart watch for display.

### CROSS-REFERENCE TO RELATED APPLICATION

The present application claims priority under 35 U.S.C. § 120 to U.S. Provisional Patent Application No. 62/855,251, filed on May 31, 2019, and entitled “Wireless Communication of Swimming Metrics,” the entire disclosure of which is incorporated by reference herein.

### BACKGROUND OF THE INVENTION

### 1. Field of Invention

This invention relates to determination and auditory communication of swimming metrics to swimmers in real-time.

### 2. Description of Related Art

Swimmers and triathletes use fitness trackers to train and meet swimming goals. As of 2018, approximately 37.6 million American adults swim at least six times per year and of that population about 9.4 million are serious lap swimmers swimming more than 50 times per year. There are about 432,000 USA Triathlon memberships (including annual and day memberships) and 4.04 million participants.

Wearable fitness trackers including smartwatches are one of the fastest growing consumer electronics markets. Fitness trackers and smartwatches have applications for swimming and can be used to track performance metrics such as SWOLF (i.e., time to swim one length plus the number of strokes for that length), pace, time, interval, stroke count, calories, stroke type, and heart rate. Information pertaining to these metrics is conveyed to a user via an integrated display, or through internal haptic feedback, i.e., vibration. However, a swimmer is unable to look at a display of a tracker or smartwatch without interrupting swimming activity. For example, a swimmer would need to stop mid-stroke or at a side of the pool to check metrics on a watch. Haptic feedback is not useful for conveying anything, but the most basic of binary information. Current wearables do not provide sufficient real-time data that swimmers and triathletes need.

Challenges with wearable fitness trackers using Bluetooth technology include disrupted and short-range communication. Classic Bluetooth radio frequency (RF) communication is unsuitable for underwater transmission because RF fails to sufficiently penetrate, travel, and propagate through water. Also, considering how Bluetooth transmission range is limited, Bluetooth is not able to transmit data continuously during swimming in open water or a large pool. Further, current Bluetooth protocols for streaming audio/music do not implement sufficient buffering. Insufficient buffering causes communication and playback interruption and loss of data as signals are dropped when, for example, a Bluetooth smartwatch on a swimmer's arm goes underwater.

The only existing solution for using a smartwatch to stream audio during swimming using Bluetooth is by removing the smartwatch from the wrist and fastening it to the head such that it is less than an inch from a wireless Bluetooth headphone. However, this approach is impractical as it prevents the use of the smartwatch in all other manners for which it was intended, e.g., visual use of display, fitness tracking, heart rate sensors, etc.

United States Patent Application Publication No. 2018/0234190 to Finnovate Group LLC, the entire disclosure of which is incorporated by reference herein, discloses a conventional wireless audio streaming system for swimmers that utilizes a combination of radio frequency and near-field magnetic induction for streaming audio from a pool-side system consisting of an electronic transmitter device and a second pool-side device such as a smartphone to a wireless, waterproof headset worn by the swimmer. This system suffers from the same drawbacks as noted above and is not practical for conveying metrics in real-time.

U.S. Pat. No. 9,655,548 to Fitbit, Inc., the entire disclosure of which is incorporated by reference herein, discloses a biometric monitoring device, i.e., a Fitbit fitness tracker, to determine swimming stroke types, number of strokes, number of swimming laps, and stroke rate. Notifications regarding swimming performance goals are conveyed to a swimmer via haptic feedback or messages conveyed on an integrated display. As noted above, this type of device suffers from the same drawbacks as noted above and is not practical for conveying audio in real-time.

U.S. Pat. No. 5,889,730 to Trigger Scuba, Inc., the entire disclosure of which is incorporated by reference herein, discloses an audio communication system to be worn by a diver that is connected to a conventional face mask, to deliver audio through a bone conductor using a transceiver configured for receiving and transmitting ultrasonic signals in water. This system suffers from the same drawbacks as noted above and is not practical for conveying metrics in real-time.

No technology exists that can communicate real-time, complete swimming metrics from a limb-worn fitness tracker or smartwatch to a swimmer in an auditory manner.

### SUMMARY OF THE INVENTION

The present invention overcomes these and other deficiencies of the prior art by providing a wireless, waterproof system for communicating swimming metrics determined by a limb-worn fitness tracker, smartwatch, and/or other sensor to and/or from ahead-worn device. The head-worn device includes an integrated headphone, a headphone jack, or a wireless headphones transceiver for communicating swimming metrics in an auditory manner. For example, the head-worn device conveys voiced performance metrics such as, but not limited to SWOLF, pace, time, interval, stroke count, calories, stroke type, distance swim, distance to buoy or destination, direction to buoy or destination, elapsed time, total swim time, course correction, and heart rate during swimming, whether in a pool or open-water, in real-time as the metrics are determined. One or more sensors in the head-worn device may be used to calculate additional or more accurate swimming metrics, which are transmitted back to the limb-worn fitness tracker or smartwatch for visual display when the swimmer stops to look at their watch.

The present invention provides a swimming metric communication system comprising a smartwatch and head-worn device, with an integrated headphone, headphone jack, and/or a wireless headphone transmitter. The system utilizes a Bluetooth Low Energy (BLE) connection between the smartwatch and head-worn device to transmit data and play audio including voiced swim metrics during a swimming activity. For example, various voiced data segments are preloaded on the head-worn device and associated with unique low-bit codes, respectively. The low-bit codes minimize the bandwidth needed. The BLE connection is used to transmit certain low-bit codes to trigger the play of respective voiced data segments. A transmission connection between the smartwatch and head-worn device, and vice-versa, is established when the swimmer's hand is out of the water (recovery) and/or when the hand passes near the face underwater (pull). Once a connection is established and codes are received, the head-worn device decodes the sequence of codes and plays through a headphone a respective voiced swimming metric without interruption to swimming activity. The head-worn device may include sensors for determining additional swim metrics, which are communicated through the same type of low-bit codes to the smartwatch, which then decodes such for display of the swim metrics.

In an embodiment of the invention, a swimming metric communications system comprises: a limb-worn device; and a head-worn device, wherein the head-worn device is configured to play one or more audio segments to a user during a swimming activity, wherein the limb-worn device transmits or receives information to or from the head-worn device via a wireless communication channel. The wireless communication channel comprises a Bluetooth low energy (BLE) communication channel. The limb-worn device is a smartwatch or fitness tracker. The head-worn device comprises an integrated headphone, a headphone jack, or a wireless headphone transmitter. The one or more audio segments comprise one or more voiced swim metrics. The head-worn device comprises memory storing the one or more voiced swim metrics. The limb-worn device comprises one or more sensors for determining the one or more swim metrics. The limb-worn device transmits one or more codes to trigger the head-worn device to play the one or more voiced swim metrics. The head-worn device comprises one or more sensors for determining a swim metric, and the limb-worn device receives a code from the head-worn device to trigger the display of the swim metric. Alternatively, the swimming metric device transmits the one or more audio segments to the head-worn device using a sequence of data packets with extended buffering protocol.

In another embodiment of the invention, a method of communicating a swimming metric to a swimmer during swimming activity, the method comprises the steps of: determining a swimming metric at a limb-worn device; transmitting, via a wireless communications channel, one or more codes associated with the determined swimming metric from the limb-worn device to a head-worn device, and playing, at the head-worn device, one or more voiced segments associated with the transmitted one or more codes. The step of transmitting is performed using a BLE communication channel. The head-worn device comprises an integrated headphone, a headphone jack, or a wireless headphone transmitter. The head-worn device comprises memory storing the one or more voiced swim metrics.

In an additional embodiment of the invention, a method of communicating a swimming metric to a swimmer during swimming activity, the method comprises the steps of: determining a swimming metric at a head-worn swimming metric device using one or more sensors; transmitting, via a wireless communications channel, one or more codes associated with the determined swimming metric from the head-worn device to the limb-worn device; and displaying, at the limb-worn device, the determined swimming metric. The wireless communication channel comprises a Bluetooth low energy (BLE) communication channel.

In yet another embodiment of the invention, a head-worn device comprises: a transceiver, wherein the transceiver communicates with a limb-worn device via a wireless communication channel; an integrated headphone, a headphone jack, or a wireless headphone transmitter; a processor; and memory including computer program code, the memory and the computer program code configured to, with the processor, cause the head-worn device to perform at least the following: receive, via the transceiver, one or more codes from the limb-worn device, and play, via the integrated headphone, the headphone jack, or the wireless headphone transmitter, to a user during a swimming activity, one or more audio files associated with the received one or more codes. The wireless communication channel comprises a Bluetooth low energy (BLE) communication channel. The one or more audio files comprise one or more voiced swimming metrics. The one or more audio files may also comprise one or more music files. The limb-worn device is a smartwatch or fitness tracker, the limb-worn device comprising one or more sensors for determining a swimming metric. The computer program code configured to, with the processor, cause the head-worn device to further perform the following: transmit, via the transceiver, one or more second codes from the head-worn device to the limb-worn device, to display the determined swimming metric at the limb-worn device.

An advantage of the invention is that it facilitates communication of real-time, swimming metrics to a swimmer in an auditory and/or display manner while swimming. Standard audio streaming via Bluetooth is unable to convey swimming metrics in real-time.

The foregoing, and other features and advantages of the invention, will be apparent from the following, more particular description of the preferred embodiments of the invention, the accompanying drawings, and the claims.

### BRIEF DESCRIPTION OF THE DRAWINGS

For a more complete understanding of the present invention and advantages thereof, reference is now made to the ensuing descriptions taken in connection with the accompanying drawings briefly described as follows.

FIG. 1 illustrates a swimming communication system according to an exemplary embodiment of the invention;

FIG. 2A illustrates a swimming communication system utilizing a head-worn device with integrated headphone according to an embodiment of the invention;

FIG. 2B illustrates a swimming communication system utilizing a head-worn device with wired headphones according to an embodiment of the invention;

FIG. 2C illustrates a swimming communication system utilizing a head-worn device with wireless headphones according to an embodiment of the invention;

FIG. 3 illustrates a swimming communication method from a smartwatch to a head-worn device according to an embodiment of the invention;

FIG. 4 illustrates a swimming communication method from a head-worn device to a smartwatch according to an embodiment of the invention;

FIG. 5 illustrates swim metric examples displayed on a smartwatch display according to an embodiment of the invention; and

FIG. 6 illustrates electronic components of a head-worn device according to an exemplary embodiment of the invention.

### DETAILED DESCRIPTION OF PREFERRED EMBODIMENTS

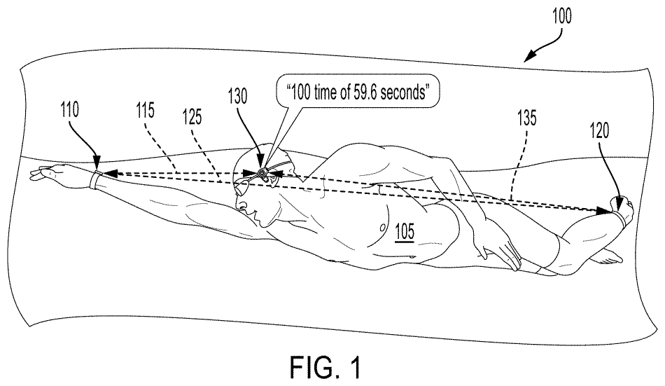

Preferred embodiments of the present invention and their advantages may be understood by referring to FIGS. 1-6, wherein like reference numerals refer to like elements. The term “head-worn device” as used herein includes any type of electronic wearable device or communications module that includes, is wired to, or wirelessly connected to a headphone, where the “headphone” as used herein has an auditory signal generator including, but not limited to a supra-aural headphone, an earbud, a canal phone, a bone conduction headphone, and a smart headphone, whether communicating unilaterally or bilaterally with a remote device. The term “swimming” as used herein refers to propelling oneself through water or treading water using one or more limbs, and water-based activities that involve swimming. In addition to lap swimming in a pool or open-water swimming, some swimming metrics such as time, calories, direction, and heart rate may be useful in other water-based activities such as, but not limited to scuba-diving, water polo, diving, water aerobics, and surfing.

In general, the present invention employs one or more sensors for determining swimming metrics during swimming. One or more sensors can be included as part of a limb-worn device such as a smartwatch or fitness tracker, a head-worn device, or other swimmer-worn device, or distributed among any combination of those devices. For purposes of the description of the present invention, the terms smartwatch and fitness tracker are used interchangeably, as a fitness tracker can include many, if not all, of the sensors included in a smartwatch. For example, sensors in a conventional smartwatch include, but are not limited to a 3-axis accelerometer, a 3-axis gyroscope, altimeter, and magnetometer, which generate raw data streams used to determine swimming metrics, the identification and implementation of which are apparent to one of ordinary skill in the art. Different smartwatches may have different available sensors. An Apple Watch Series 4, for example, includes a global positioning system (GPS), barometric altimeter, electrical heart sensor, optical heart sensor, accelerometer, gyroscope, and an ambient light sensor. These and other sensors such as, but not limited a 3-axis accelerometer, a 3-axis gyroscope, altimeter, and magnetometer can be included as part of the head-worn device of the present invention. The term smartwatch refers to any type of wearable computer worn on a limb.

The present invention further includes software, e.g., an app, executing at one or more of the above-noted devices. In an embodiment of the invention, the app employs classifiers derived from machine learning algorithms operating on data sets recorded with different swimmers. Classifiers convert the raw data streams from sensors into features (e.g., rotation rates, roll, pitch, yaw, peak accelerations, short-term displacement, and data statistics such as moving mean, standard deviation, max-min, etc.), which are used as input to detect (i.e., classify) a swimming stroke (e.g., butterfly, back, breast, free), turn, or rest. A Markov state-machine is used to restrict the class and class-transition, the implementation of which is apparent to one of ordinary skill in the art. For example, if a swimmer is at rest, then he/she can only transition to swimming or stay at rest. Once swimming, a different classifier is used to determine the stroke. From swimming, one can transfer to a turn, stop, or continue swimming. Median and majority-vote smoothing of the classifier output is used to remove outliers and enforce additional heuristic rules (e.g., swimming needs to take place for longer than 10 seconds in a 25 yard pool; if breaststroke and butterfly are alternately detected during a lap, then the majority stroke is decided; rest has a minimum of 2 seconds otherwise it is considered a turn, etc.). Several different classifier types have been evaluated and may be used including logistic regression, support vector machines, and Gaussian Mixture Models, the implementation of which are apparent to one of ordinary skill in the art. In addition to the heuristic rules, a Hidden-Markov-Model with Viterbi decoding could be used to more optimally enforce transitions through the state-machine, the implementation of which is apparent to one of ordinary skill in the art.

The app processes various sensor data streams, features, and/or classifier output to determine swimming metrics such as, but not limited to SWOLF, pace, interval, stroke count, calories, stroke type (e.g., butterfly, backstroke, breaststroke, freestyle), drill (e.g., kicking), heart rate, lap count, lap time, split time, swim time, stroke cycles, distance swam, distance to buoy or destination, direction to buoy or destination, elapsed time, total swim time, head position, breath count, and course correction. In an embodiment of the invention, the app utilizes a publicly available application programming interface to interact with a respective, proprietary operating system provided with the smartwatch to determine one or more of the above-noted metrics, or to acquire additional swimming metrics, provided by proprietary algorithms running on the smartwatch.

FIG. 1 illustrates a swimming communication system 100 according to an embodiment of the invention. The swimming communication system 100 comprises a smartwatch 110, an optional swimming metric device 120, and a head-worn device 130. For purposes of illustrations, the head-worn device 130 is not drawn to scale; actual physical size may be smaller. The smartwatch 110 executes an app as noted above and communicates with the head-worn device 130 via a wireless communications channel 115. The optional swimming metric device 120 is provided to improve accuracy of swimming metrics calculation. For example, a sensor placed on a leg of a swimmer better facilitates tracking of laps count during kicking drills, kick rate, and turns. The device 120 communicates with the smartwatch 110 via a wireless communications channel 125, which enables transfer of sensor data, features, and/or classifier output from the device 120 to the smartwatch 110. The device 120 may also communicate with the head-worn device via a wireless communications channel 135, which enables transfer of sensor data, features, and/or classifier output from the device 120 to the head-worn device 130. In an embodiment of the invention, the wireless communications channels 115, 125, and 135 utilize Bluetooth Low Energy (Bluetooth LE or BLE). The head-worn device 130 includes a clip (not shown) for attaching the device 130 to the swimmer's googles, a head strap, a swim cap, swim mask, ear, or hair.

The head-word device 130 includes a processing unit, a BLE transceiver, memory, and a power source, e.g., rechargeable battery. The head-worn device 130 further includes an integrated headphone, a headphone jack for a wired headphone, and/or a transceiver for a wireless headphone. Real-time swim metrics for pool or open swimming are auditorily communicated to the user 105 via the head-worn device 130 via one of these headphones. In an embodiment of the invention, various swim metrics are heard by the user 105 as spoken phrases, i.e., voiced swim metrics. For example, as shown in FIG. 1, a certain swim metric is played as a voiced metric “100 time of 59.6 seconds” directly to the swimmer. Other phrases are used to communicate other metrics. In general, the smartwatch 110 determines swim metrics and transmits data related to the various voiced swim metrics to be communicated. However, to preserve bandwidth and to prevent audio drop-outs over the communications channel 115, various predetermined phrases may be recorded and stored at the head-worn device with the head-worn device 130, which are triggered for playback upon reception of respective low-bit rate codes, e.g., 16 bits or less, received over the channel 115. The implementation of low-bit rate codes is explained in detail below.

Data transmission over the channel 115 occurs during a cycle of the stroke when both the smartwatch 110 and head-worn device 130 are out of the water or within a few inches of the surface of the water allowing for a wireless data transmission. A secure connection on channel 115 can be made, for example, when the user's hand is out of the water (recovery), during the underwater pull when the hand passes near the face (pull), or both.

In an embodiment of the invention, the head-worn device 130 comprises one or more sensors as identified above for improved accuracy of swimming metrics calculations. For example, a sensor located on the head provides accurate timing of lap and split times, or real-time feedback on head position, e.g., angle of head during backstroke, or counting number of breathes. The one or more sensors can be a 3-axis accelerometer, a 3-axis gyroscope, a magnetometer, another type of sensor readily apparent to one of ordinary skill in the art, or a combination thereof. In an embodiment of the invention, the swimming metrics determined from the sensors on the head-worn device 130 are transmitted to the smartwatch 110 allowing for visual review of the swimming metrics when the swimmer stops to look at their smartwatch.

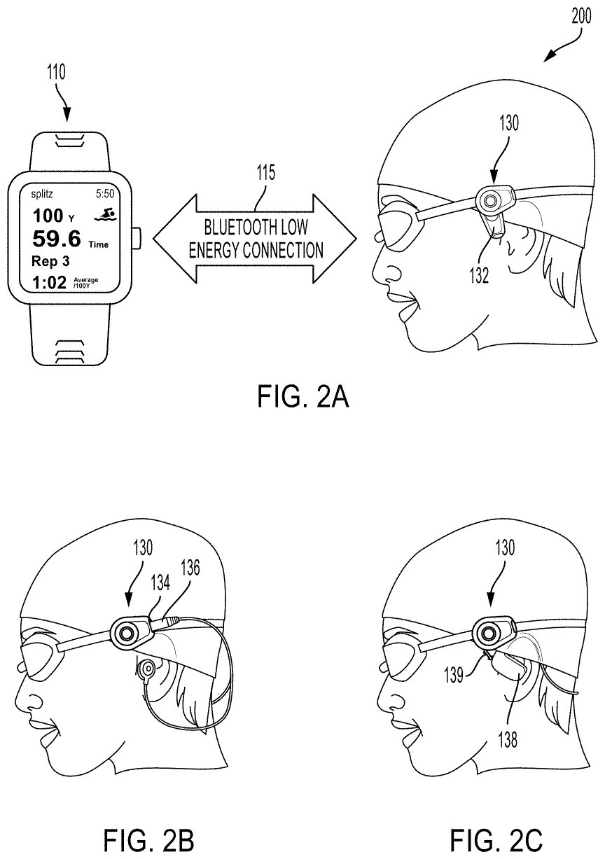

FIG. 2A illustrates a swimming communication system 200 according to an embodiment of the invention. The smartwatch 110 is worn on the wrist. Here, head-worn device 130 comprises an integrated headphone such as bone-conduction headphone attachment 132. In another exemplary embodiment of the invention, as shown in FIG. 2B, the head-worn device 130 comprise an audio jack 134 for use with wired headphones 136, the wired headphones being either supra-aural, e.g. earbuds, as shown, or bone conduction, the identification and implementation of which are apparent to one of ordinary skill in the art. Referring to FIG. 2C, in another exemplary embodiment of the invention, the head-worn device 130 may further comprise a wireless, e.g., Bluetooth, transceiver for use with wireless headphones 138, the identification and implementation of which is apparent to one of ordinary skill in the art. Because the distance 139 between the head-worn device 130 and a wireless headphone 138 can be positioned within inches, standard Bluetooth can be used to maintain uninterrupted connectivity during swimming (unlike the communication channel 115 between then head-worn device 130 and the limb-worn smartwatch 110). The headphones 136 (or wireless headphones 138) can be a single ear unit or two ear units that play stereo audio. The head-worn device 130 can be attached to a goggle strap, worn on the back of the head, worn on the side of the head, or secured on the ear.

As shown, the ring on the head-worn device 130 is an LED light with a button in the center. The LED is used to indicate charging, low power, and for initial pairing with the smartwatch 110, the optional limb-worn device 120, and/or wireless headphones 138. The button is used for actions such as, but not limited to pairing, turning the device 130 on/off, replay of the last voiced metric, playing of additional voiced metrics (e.g., elapsed workout time and total distance), or for controlling music playback. A ring is just one example of how the LED light and button would look; other spatial configurations and form factors of the head-worn device may be utilized. The button may also include a touch sensor for gesture control and/or biometric authentication, the implementation of which are apparent to one of ordinary skill in the art.

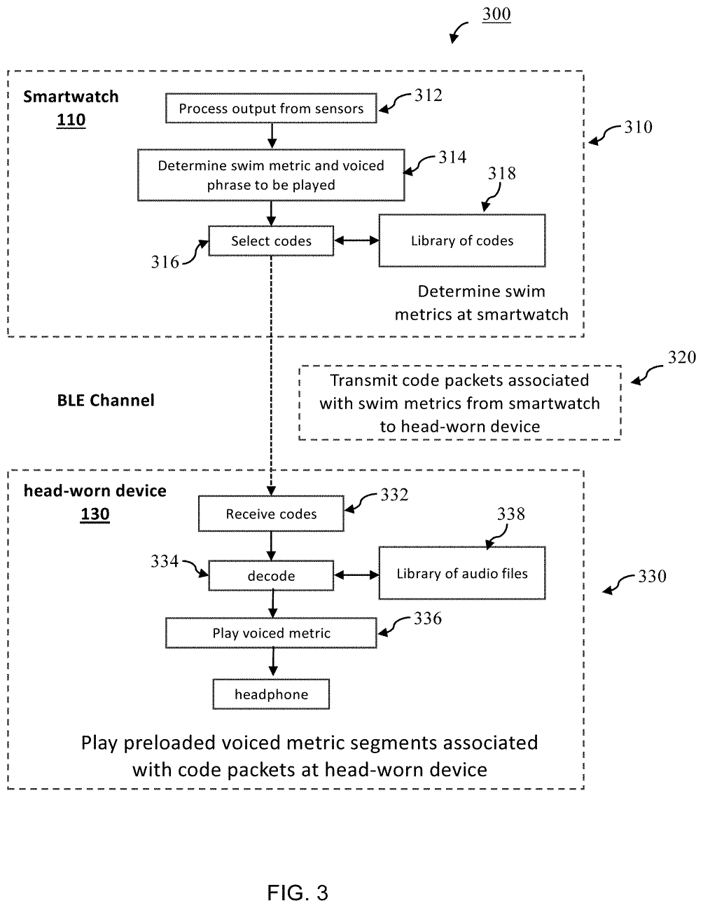

FIG. 3 illustrates a swimming communication method 300 according to an embodiment of the invention. The app on the smartwatch 110 determines (step 310) one or more swim metrics to convey to the user 105. Particularly, the app processes (step 312) the output of the one or more sensors in the smartwatch 110 as noted above and determines (step 314) a swim metric and voiced phrases to be played. The app selects the codes (step 316) associated with the swim metric and voiced phrases to be played from the library of codes 318. The smartwatch 110 then sends (step 320) code packets, i.e., a sequence of one or more codes associated with the determined swim metrics, during the swim cycle when both it and the head-worn device 130 are out of the water or within a few inches of the surface of the water to enable a secure BLE connection. BLE is adequate to transmit low-bit rate codes instead of streaming data. Various voiced metric segments are preloaded as a library of audio files 338 in memory on the head-worn device 130. The smartwatch 110 essentially acts as an automatic remote control to specify playback of a certain group of the preloaded voiced metric segments from the preloaded library of audio files 338. For example, the smartwatch triggers segments “50,” “split of,” “32,” “0.4,” and “seconds,” through the transmission of respective 16-bit binary codes (code packets selected from a predefined library of codes), to trigger playback of “fifty, split of thirty-two-point-four seconds” to the user 105. The code packets can be of any length; however, 16-bit codes provide 65,536 unique codes while maintaining a relatively short length. Numerous segments related to swim metrics can be preloaded, the identification of which is apparent to one of ordinary skill in the art. Each segment is associated with a respective unique binary code, the length of which is sufficient to encompasses all possible preloaded segments.

The head-worn device 130 plays (step 330) preloaded voice metric segments associated with the received code packets. Particularly, the head-worn device 130 receives (step 332) the code packets sent by the smartwatch 110. Then, the head-worn device 130 decodes (334) the received code packets to identify the audio files associated with the received code packets from the preloaded library of audio files 338. The head-worn device 130 then plays (step 336) the identified audio files through the headphone 132, 136, or 138.

When Bluetooth and BLE devices communicate, one must be designated as the “peripheral” and the other as a “central.” In one embodiment, the smartwatch 110 is the central and the head-worn device 130 is the peripheral. Currently, WatchOS restricts the Apple Watch to be a central when communicating with other devices. In this configuration, the peripheral advertises itself and waits for the central to connect to it. Once “connected,” data can be communicated in either direction. A BLE 4.2 (or subsequent versions such as, but not limited to 5.0, 5.1, and 5.2) connection is capable of re-connection and transmission within 0.1 seconds, which permits transmission of various 16-bit codes necessary to specify a voiced phrase about a swim metric (e.g., lap count or split time) at the first arm stroke after pushing off from the wall (side of the pool). To achieve a seamless, uninterrupted BLE re-connection, it is necessary to have the head-worn device 130 configured as a peripheral continually “advertise” its availability to connect. The software on the smartwatch 110 configured as a central uses a control loop or timer to continuously check for the availability of the head-worn device 130. The smartwatch 110 can also keep a BLE “profile” in its memory so that BLE reconnection with the head-worn device 130 is achieved more quickly without having to “rediscover” all the attributes of the BLE connection on each re-connection. The head-worn device 130 advertises itself and waits for the smartwatch 110 to connect to it. Once “connected,” data is transmitted between the devices. The smartwatch 110 can either be worn on the wrist or replaced with a fitness tracker or other swimming metric device worn elsewhere on the body.

In another embodiment, designation of the central and peripheral are reversed, i.e., the smartwatch 110 is the peripheral and the head-worn device 130 in the central. The advantage of this configuration is that a smartwatch designated as the peripheral may “broadcast” both its availability to connect concurrently with code packets, i.e., one or more codes as noted above. The head-worn device 130 designated as the central can receive the broadcasted codes without having to establish a connection (or re-connection). Effectively, the peripheral device (e.g., the smartwatch 110) broadcasts codes and the central device (e.g., head-worn device 130) just listens for the codes. This configuration should be more robust to intermittent disruptions, such as the hand going underwater, by alleviating the need for a full re-connection before transmitting the codes associated with swim metrics.

Communications between the smartwatch 110 and the head-worn device 130 requires BLE transmission of codes to specify a swimming metric. For this reason, the earliest played voiced metric occurs during the first stroke after a turn (not while pushing off underwater). The smartwatch 110 has sensors to calculate swimming metrics. One method of calculating swimming metrics involves extracting raw data from swimming metric sensors and data from proprietary algorithms to calculate the metrics. A second method involves the smartwatch calculating swimming metrics using operations built-in to the operating system and providing such metrics through the smartwatch API. These swimming metrics include stroke, swim duration, stoke count, lap time, etc. However, because a smartwatch 110 (e.g., Apple Watch) might not be intended to provide real-time feedback during swimming activity, some metrics such as lap count, lap time, etc. are only available with a latency several seconds after recording the swimming activity. Therefore, the present swimming metric communication system overcomes this limitation by extracting swimming metrics either on the smartwatch 110 or on a sensor in the head-worn device 130, and/or the limb-worn device 120.

While a voiced metric is transmitted and played audibly on the first stroke after pushing off from the wall; voiced metrics cannot be played during the underwater push-off because both the smartwatch 110 and the head-worn device 130 must be in close range underwater, or out of or near the surface of the water during the same time to establish a data connection. For example, in another embodiment of the invention, this limitation is overcome by adding sensors (e.g., inertial and magnetometer) and a processor to the head-worn device 130 to detect the timing of turns, starts and stops. This enables the determination of the split and lap count metrics to be calculated directly on the head-worn device 130 such that the voiced metrics could be played sooner (e.g., during the underwater push-off instead of after an arm stroke), or for use with all swim strokes (including breaststroke and backstroke). The location and addition of sensors and processing at the head-worn device 130 achieves faster response and greater accuracy than using sensors on the wrist for select swim metrics such as, but not limited to lap time, split time, breath count, and head position. The smartwatch 110 still serves an important integrated role. Sensors and processing on the smartwatch 110 are still used to extract less time-critical metrics such as total swim duration, total distance, stroke type, stroke count, and open water swim metrics using its global positioning system (GPS) for distance or direction to a buoy or destination. These metrics are transmitted to the head-worn device 130, but not necessarily immediately after a turn. The smartwatch 110 may also be used for a visual user interface, for controlling various settings on the head-worn device 130, and for remote control of music (e.g., fast forward, stop, start, volume control), and for displaying swimming metrics that are calculated using sensors on the smartwatch 110 or from sensors on the head-worn device 130 as noted above, and/or optional limb-worn device 120. These visual display functions occur when the swimmer is resting or not swimming where a continuous BLE data connection is present.

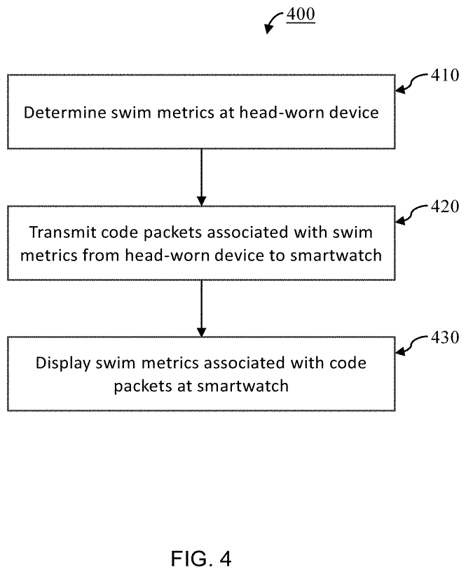

FIG. 4 illustrates a swimming communication method 400 according to an embodiment of the invention. Here, swim metrics are determined (step 410) at the head-worn device 130 using one or more sensors at the head-worn device 130 as described above. The sensors in the head-worn device 130 may be used separately from the sensors in the smartwatch 110 to determine a swim metric. Alternatively, the head-worn device 130 determines a swim metric by utilizing its sensors to refine or improve a swim metric originally determined by the sensors in the smartwatch 110. Either way, the determined swim metric at the head-worn device 130 is communicated (step 420) to the smartwatch 110 through the transmission of code packets as noted above. The swim metric is then displayed (step 430) at the smartwatch or conveyed to the user 105 through haptic feedback.

In another embodiment of the invention, instead of storing voiced segments on the head-worn device 130 prior to swimming, a custom extended buffering protocol is used to stream voiced phrases from the smartwatch 110 to the head-worn device with head-worn device 130. Because the conventional way of transmitting audio files with Bluetooth results in unacceptable interruptions when the smartwatch 110 is underwater, extra buffering is done in software both on the smartwatch 110 and the head-worn device 130. For an audio file to be transmitted (or streamed) from the smartwatch 110, the software first buffers the data in memory. This data is then segmented into data packets and transmitted using the BLE protocol established and described above (connection during the cycle of the stroke when the arm is out of the water). Standard error-correcting techniques can also be used to verify a data packet has been received, otherwise the data packet can be resent to avoid missed data packets. The BLE bandwidth and data rates allows audio data to be transmitted in faster-than real time during short connection periods (e.g., an approximately 0.25 second-connection time allows for approximately 1-4 seconds of audio data to be transmitted). The head-worn device 130 receives the data packets, stuffs them in a buffer (memory), and then waits until all the data corresponding to a complete voiced metric phrase had been fully transmitted, at which point it can then play the audio continuously. For transmission of streaming music, the buffer would need to be several seconds long or longer. Playing of music is then delayed by several seconds relative to the streaming data coming from the smartwatch 110. The main advantage of this method is that it could allow for the transmission of streaming music services (e.g., Spotify or Pandora) from the smartwatch 110 to the head-worn device 130 during swimming.

In another embodiment of the invention, the smartwatch 110 comprises a wristband designed as a “relay” device for underwater transmission. Specific smartwatches allow for the use of interchangeable bands (usually for fashion). A custom band integrates an electronic device capable of maintaining a constant Bluetooth connection with the smartwatch even underwater due to its close proximity. The smartwatch band then acts as a “relay,” receiving data through Bluetooth from the smartwatch 110 and then using a different transmitter capable of underwater communications such as, but not limited to NFMI, modulated light diode, or low-frequency RF to send data to the head-worn device. In another embodiment of the invention, the wristband includes a Bluetooth antenna for a better Bluetooth connection of the smartwatch 110 to the head-worn device 130.

As noted above, swim metrics can be determined with sensors on the smartwatch 110, sensors on the head-worn device 130, sensors in the optional swim metric device 120, or any combination thereof. Open-water metrics such as pace, distance and direction to a buoy can be extracted from a GPS on the smartwatch 110.

Current swim tracking on conventional smartwatches does not provide lap count during kick sets, i.e., swimming using only the legs and feet for propulsion without arm rotation. This tends to be one of the biggest complaints of users, as swimmers miss out on tracking their yardage during the kicking part of a workout. The reason is likely that a smartwatch detects swimming based on arm stroke/movement and kicking is simply classified as resting (when there is no arm movement). In an embodiment of the invention, detecting laps during kicking is provided as follows: first, a machine-learning classifier (similar approach to above) classifies the difference between swimming, rest, and kicking. If the confidence of kicking is above a minimum threshold, then laps are detected as follows. If the pool is outdoors and a GPS signal can be acquired, then positioning can be used to estimate distance traveled, direction changes, and time between direction changes. This information along with a set of rules (e.g. minimal distance traveled and minimal time between direction changes must fall within some range) to determine lap count, assuming the pool length is known. For indoor pools (where GPS is not available), an added magnetometer on the head-worn device 130 may be used in combination with inertial sensors. The magnetometer is used to determine direction changes indicating a possible lap change, while the inertial sensors are used to indicate motion.

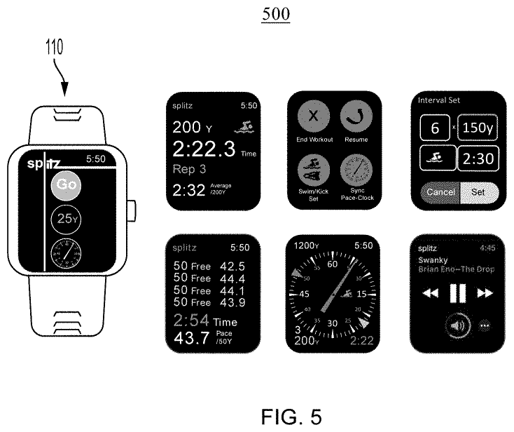

FIG. 5 illustrates exemplary swim metrics and other information displayed on a smartwatch display. For example, displays 500 convey information such as swim distance, music player, heart rate, swim strokes, pace, swim time, lap time, elapsed swim time, music track, options for voiced swim metrics, etc. Other swim metrics or data not shown, but apparent to one of ordinary skill in the art, may also be displayed. A chosen subset of displayed swim metrics can be communicated to the swimmer through communication channel 115 as voided metrics to the swimmer via the head-worn device 130.

In another embodiment of the invention, audio files are stored on the head-worn device 130 for music playback or guided workouts. Software on the smartwatch 110 sends the above-noted codes or code packets to trigger playback of the audio files. For example, audio files stored on the head-worn device 130 are played in a sequence to instruct a user to perform certain swimming exercises. For music playback, the app on the smartwatch 110 can be used to play, pause, skip songs, shuffle songs, and adjust volume.

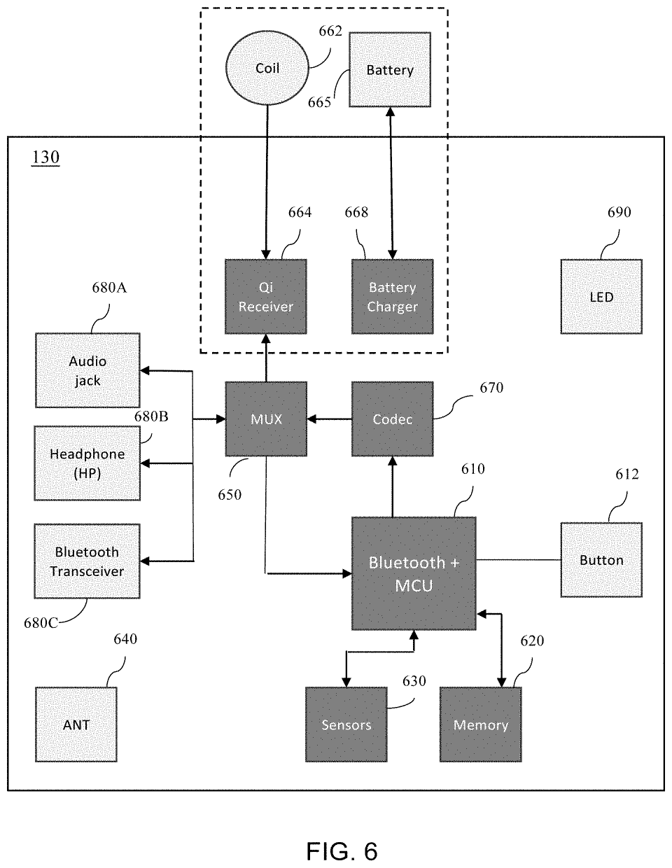

FIG. 6 illustrates electronic components of the head-worn device 130 according to an exemplary embodiment of the invention. The head-worn device 130 comprises an embedded system on a chip (SoC) 610, which combines a Bluetooth transceiver and a microcontroller unit (MCU) including or more processors configured to execute computer program code stored in memory. The SoC 610 is coupled to flash memory 620 for storing audio files, and one or more inertial and magnetic sensors providing six or nine degrees of freedom 530, a Bluetooth antenna 640, a multiplexer 650, power source 660, codec 670 used for audio file decoding and driving a headphone jack 680A and/or integrated headphone 680B, which together with the computer program code cause the head-worn device 130 to perform the various activities noted above. Alternatively, a second Bluetooth transceiver 680C permits connection to a wireless headphone as noted above. The headphone jack 680A can also be used for wired charging and/or uploading of audio files as well. The power source 660 comprises a receiver coil 662, receiver Qi 664, and battery charger 665 for wireless charging of a battery 668, the implementation of all of which is apparent to one of ordinary skill in the art. An LED 690 is provided to denote battery charging status and Bluetooth pairing status with the smartwatch 110 and/or wireless headphones as noted above. A button 612 is provided as a user-interface in order to, among other things, turn on/off the head-worn device 130, initiate Bluetooth pairing with the smartwatch 110, and initiate Bluetooth pairing with the wireless headphones as noted above.

The invention has been described herein using specific embodiments for the purposes of illustration only. It will be readily apparent to one of ordinary skill in the art, however, that the principles of the invention can be embodied in other ways. Therefore, the invention should not be regarded as being limited in scope to the specific embodiments disclosed herein, but instead as being fully commensurate in scope with the following claims.

1. A swimming metric communications system comprising:
a limb-worn device; and
a head-worn device, wherein the head-worn device is configured to play one or more audio segments to a user during a swimming activity, wherein the limb-worn device transmits or receives information to or from the head-worn device via a wireless communication channel.

2. The system of claim 1, wherein the wireless communication channel comprises a Bluetooth low energy (BLE) communication channel.

3. The system of claim 1, wherein the limb-worn device is a smartwatch or fitness tracker.

4. The system of claim 1, wherein the head-worn device comprises an integrated headphone, a headphone jack, or a wireless headphone transmitter.

5. The system of claim 1, wherein the one or more audio segments comprise one or more voiced swim metrics.

6. The system of claim 5, wherein the head-worn device comprises memory storing the one or more voiced swim metrics.

7. The system of claim 6, wherein the limb-worn device comprises one or more sensors for determining the one or more voiced swim metrics.

8. The system of claim 7, wherein the limb-worn device transmits one or more codes to trigger the head-worn device to play the one or more voiced swim metrics.

9. The system of claim 1, wherein the head-worn device comprises one or more sensors for determining a swim metric, and the limb-worn device receives a code from the head-worn device to trigger the display of the swim metric.

10. The system of claim 1, wherein the swimming metric device transmits the one or more audio segments to the head-worn device using a sequence of data packets with extended buffering protocol.

11. A method of communicating a swimming metric to a swimmer during swimming activity, the method comprising the steps of:
determining a swimming metric at a limb-worn device;
transmitting, via a wireless communications channel, one or more codes associated with the determined swimming metric from the limb-worn device to a head-worn device, and
playing, at the head-worn device, one or more voiced segments associated with the transmitted one or more codes.

12. The method of claim 11, wherein the step of transmitting is performed using a BLE communication channel.

13. The method of claim 11, wherein the head-worn device comprises an integrated headphone, a headphone jack, or a wireless headphone transmitter.

14. The method of claim 11, wherein the head-worn device comprises memory storing the one or more voiced swim metrics.

15. A method of communicating a swimming metric to a swimmer during swimming activity, the method comprising the steps of:
determining a swimming metric at a head-worn swimming metric device using one or more sensors;
transmitting, via a wireless communications channel, one or more codes associated with the determined swimming metric from the head-worn device to the limb-worn device; and
displaying, at the limb-worn device, the determined swimming metric.

16. The method of claim 15, wherein the wireless communication channel comprises a Bluetooth low energy (BLE) communication channel.

17. A head-worn device comprising:
a transceiver, wherein the transceiver communicates with a limb-worn device via a wireless communication channel;
an integrated headphone, a headphone jack, or a wireless headphone transmitter;
a processor; and
memory including computer program code, the memory and the computer program code configured to, with the processor, cause the head-worn device to perform at least the following:
receive, via the transceiver, one or more codes from the limb-worn device, and
play, via the integrated headphone, the headphone jack, or the wireless headphone transmitter, to a user during a swimming activity, one or more audio files associated with the received one or more codes.

18. The head-worn device of claim 17, wherein the wireless communication channel comprises a Bluetooth low energy (BLE) communication channel.

19. The head-worn device of claim 17, wherein the one or more audio files comprise one or more voiced swimming metrics.

20. The head-worn device of claim 17, wherein the one or more audio files comprise one or more music files.

21. The head-worn device of claim 17, wherein the limb-worn device is a smartwatch or fitness tracker, the limb-worn device comprising one or more sensors for determining a swimming metric.

22. The head-worn device of claim 17, wherein the computer program code configured to, with the processor, cause the head-worn device to further perform the following:
transmit, via the transceiver, one or more second codes from the head-worn device to the limb-worn device, to display the determined swimming metric at the limb-worn device.
